(19) United States
(12) Patent Application Publication
(10) Pub. No.: US 2010/0048548 A1
(43) Pub. Date: Feb. 25, 2010

(54) IMINO-OXAZOLIDINES AND USE THEREOF

(75) Inventors: Susanne Röhrig, Essen (DE); Jens Pohlmann, Basel (CH); Elisabeth Perzborn, Wuppertal (DE); Christoph Gerdes, Leverkusen (DE); Karl-Heinz Schlemmer, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer HealthCare AG, Leverkusen (DE)

(21) Appl. No.: 11/919,020

(22) PCT Filed: Apr. 8, 2006
(86) PCT No.: PCT/EP2006/003232

§ 371 Date: Nov. 9, 2009

(30) Foreign Application Priority Data
Apr. 22, 2005 (DE) 10 2005 018 690.4

(51) Int. Cl. A61K 31/422; C12N 5/00; A61K 31/5377; A61K 31/506; A61K 31/445; A61K 31/44; C07D 413/14; C07D 413/10; A61P 7/00; A61P 7/02

(52) U.S. Cl. 514/230.8; 435/375; 514/274; 514/326; 514/340; 514/376; 544/96; 544/137; 544/316; 546/271.4; 548/229

(57) ABSTRACT

The present invention relates to novel iminooxazolidines, to processes for their preparation, to their use for the treatment and/or prophylaxis of diseases and also to their use for preparing medicaments for the treatment and/or prophylaxis of diseases, in particular thromboembolic disorders.

The present invention relates to novel iminooxazolidines, to processes for their preparation, to their use for the treatment and/or prophylaxis of diseases and also to their use for preparing medicaments for the treatment and/or prophylaxis of diseases, in particular thromboembolic disorders.

Blood coagulation is a protective mechanism of the organism which helps to “seal” defects in the wall of the blood vessels quickly and reliably. Thus, loss of blood can be avoided or kept to a minimum. Haemostasis after injury of the blood vessels is effected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a joint reaction path, are distinguished. Here factor Xa, which is formed from the proenzyme factor X, plays a key role, since it connects the two coagulation paths. The activated serine protease Xa cleaves prothrombin to thrombin. The resulting thrombin, in turn, cleaves fibrinogen to fibrin. Subsequent crosslinking of the fibrin monomers causes formation of blood clots and thus haemostasis. In addition, thrombin is a potent effector of platelet aggregation which likewise contributes significantly to haemostasis.

Haemostasis is subject to a complex regulatory mechanism. Uncontrolled activation of the coagulant system or defective inhibition of the activation processes may cause formation of local thrombi or embolisms in vessels (arteries, veins, lymph vessels) or in heart cavities. This may lead to serious thromboembolic disorders. In addition, in the case of consumption coagulopathy, hypercoaguability may—systemically—result in disseminated intravascular coagulation. Thromboembolic complications furthermore occur in microangiopathic haemolytic anaemias, extracorporeal blood circulation, such as haemodialysis, and also in connection with prosthetic heart valves.

Thromboembolic disorders are the most frequent cause of morbidity and mortality in most industrialized countries. The incidence of venous thromboembolisms (VTE) is estimated to be higher than 1 case per 1000 persons [R. H. White, “The epidemiology of venous thromboembolism” Circulation 2003, 107 (Suppl. 1), 14-18]. Every year, about 1.3 to 4.1 of 1000 persons suffer a first stroke [V. L. Feigin, C. M. Lawes, D. A. Bennett, C. S. Anderson, Lancet Neurol. 2003, 2, 43-53] and about 5 of 1000 persons suffer a myocardial infarction [J. Fang, M. H. Alderman, Am. J. Med. 2002, 113, 208-214].

The anticoagulants, i.e. substances for inhibiting or preventing blood coagulation, which are known from the prior art, have various, often grave disadvantages. Accordingly, in practice, an efficient treatment method or prophylaxis of thromboembolic disorders is very difficult and unsatisfactory.

In the therapy and prophylaxis of thromboembolic disorders, use is firstly made of heparin, which is administered parenterally or subcutaneously. Owing to more favourable pharmacokinetic properties, preference is nowadays more and more given to low-molecular-weight heparin; however, even with low-molecular-weight heparin, it is not possible to avoid the known disadvantages described below, which are involved in heparin therapy. Thus, heparin is ineffective when administered orally and has a relatively short half-life. Since heparin inhibits a plurality of factors of the blood coagulation cascade at the same time, the action is nonselective. Moreover, there is a high risk of bleeding; in particular, brain haemorrhages and gastrointestinal bleeding may occur, which may result in thrombopenia, drug-induced alopecia or osteoporosis [Pschyrembel, Klinisches Wörterbuch, 257th edition, 1994, Walter de Gruyter Verlag, page 610, entry “Heparin”; Römpp Lexikon Chemie, Version 1.5, 1998, Georg Thieme Verlag Stuttgart, entry “Heparin”].

A second class of anticoagulants are the vitamin K antagonists. These include, for example, 1,3-indanediones, and especially compounds such as warfarin, phenprocoumon, dicumarol and other coumarin derivatives which inhibit the synthesis of various products of certain vitamin K-dependent coagulation factors in the liver in a non-selective manner. Owing to the mechanism of action, however, the onset of the action is very slow (latency to the onset of action 36 to 48 hours). It is possible to administer the compounds orally; however, owing to the high risk of bleeding and the narrow therapeutic index, a time-consuming individual adjustment and monitoring of the patient are required [J. Hirsh, J. Dalen, D. R. Anderson et al., “Oral anticoagulants: Mechanism of action, clinical effectiveness, and optimal therapeutic range” Chest 2001, 119, 8S-21S; J. Ansell, J. Hirsh, J. Dalen et al., “Managing oral anticoagulant therapy” Chest 2001, 119, 22S-38S; P. S. Wells, A. M. Holbrook, N. R. Crowther et al., “Interactions of warfarin with drugs and food” Ann. Intern. Med. 1994, 121, 676-683].

Recently, a novel therapeutic approach for the treatment and prophylaxis of thromboembolic disorders has been described. This novel therapeutic approach aims to inhibit factor Xa. Because of the central role which factor Xa plays in the blood coagulation cascade, factor Xa is one of the most important targets for anticoagulants [J. Hauptmann, J. Stürzebecher, Thrombosis Research 1999, 93, 203; S. A. V. Raghavan, M. Dikshit, “Recent advances in the status and targets of antithrombotic agents” Drugs Fut. 2002, 27, 669-683; H. A. Wieland, V. Laux, D. Kozian, M. Lorenz, “Approaches in anticoagulation: Rationales for target positioning” Curr. Opin. Investig. Drugs 2003, 4, 264-271; U. J. Ries, W. Wienen, “Serine proteases as targets for antithrombotic therapy” Drugs Fut. 2003, 28, 355-370; L.-A. Linkins, J. I. Weitz, “New anticoagulant therapy” Annu. Rev. Med. 2005, 56, 63-77].

It has been shown that, in animal models, various both peptidic and nonpeptidic compounds are effective as factor Xa inhibitors. A large number of direct factor Xa inhibitors is already known [J. M. Walenga, W. P. Jeske, D. Hoppensteadt, J. Fareed, “Factor Xa Inhibitors Today and beyond” Curr. Opin. Investig. Drugs 2003, 4, 272-281; J. Ruef, H. A. Katus, “New antithrombotic drugs on the horizon” Expert Opin. Investig. Drugs 2003, 12, 781-797; M. L. Quan, J. M. Smallheer, “The race to an orally active Factor Xa inhibitor: Recent advances” Curr. Opin. Drug Discovery & Development 2004, 7, 460-469]. Factor Xa inhibitors having an oxazolidinone partial structure are described in WO 01/47919, WO 02/064575 and WO 03/000256.

It is an object of the present invention to provide novel substances for controlling disorders, in particular thromboembolic disorders.

The present invention provides compounds of the general formula (I)

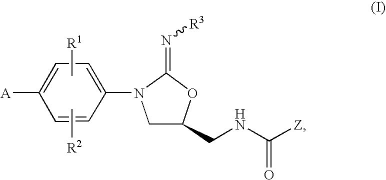

in which
A represents a group of the formula

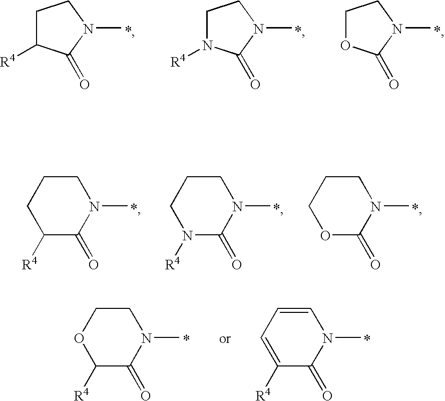

in which
R⁴ represents hydrogen, (C₁-C₆)-alkyl, (C₃-C₇)-cycloalkyl, hydroxy, (C₁-C₆)-alkoxy, amino, mono- or di-(C₁-C₆)-alkylamino, (C₃-C₇)-cycloalkylamino, (C₁-C₆)-alkanoylamino or (C₁-C₆)-alkoxycarbonylamino, where
   (C₁-C₆)-alkyl, (C₁-C₆)-alkoxy, mono- and di-(C₁-C₆)-alkylamino for their part may in each case be substituted by hydroxyl, (C₁-C₄)-alkoxy, amino, mono- or di-(C₁-C₄)-alkylamino, (C₃-C₇)-cycloalkylamino or a 4- to 7-membered saturated heterocycle which is attached via a nitrogen atom and which may contain a ring member from the group consisting of N—R⁵ and O, where
   R⁵ represents hydrogen or (C₁-C₄)-alkyl,
and * denotes the point of attachment to the phenyl ring,
or
A represents a group of the formula —C(═O)—NR⁶R⁷, where
   R⁶ and R⁷ are identical or different and independently of one another are (C₁-C₆)-alkyl or (C₃-C₇)-cycloalkyl
   or
   R⁶ and R⁷ together with the nitrogen atom to which they are attached form a 4- to 6-membered saturated or partially unsaturated heterocycle which may contain one ring member from the group consisting of N—R⁸ and O and which may be substituted by (C₁-C₆)-alkyl, hydroxy, (C₁-C₆)-alkoxy, oxo, amino, mono- or di-(C₁-C₆)-alkylamino, where
      R⁸ represents hydrogen or (C₁-C₆)-alkyl,
      where for their part all (C₁-C₆)-alkyl groups mentioned may be substituted by hydroxyl, (C₁-C₄)-alkoxy, amino, mono- or di-(C₁-C₄)-alkylamino or (C₃-C₇)-cycloalkylamino,
Z represents phenyl, pyridyl, pyrimidinyl, pyrazinyl, thienyl, furyl or pyrrolyl which may in each case be mono- or disubstituted by identical or different substituents selected from the group consisting of halogen, cyano, (C₁-C₄)-alkyl, which for its part may be substituted by hydroxyl or amino, ethynyl, cyclopropyl and amino,
R¹ and R² are identical or different and independently of one another represent hydrogen, halogen, cyano, (C₁-C₄)-alkyl, cyclopropyl, trifluoromethyl, hydroxyl, (C₁-C₄)-alkoxy, trifluoromethoxy, amino, mono- or di-(C₁-C₄)-alkylamino, where
   (C₁-C₄)-alkyl and (C₁-C₄)-alkoxy for their part may in each case be substituted by hydroxyl, (C₁-C₄)-alkoxy, amino, mono- or di-(C₁-C₄)-alkylamino or (C₃-C₇)-cycloalkylamino,
and
R³ represents hydrogen, (C₁-C₆)-alkyl or cyano,
and salts, solvates and solvates of the salts thereof.

Compounds according to the invention are the compounds of the formula (I) and their salts, solvates and solvates of the salts, the compounds, comprised by formula (I), of the formulae mentioned below and their salts, solvates and solvates of the salts and the compounds, comprised by the formula (I), mentioned below as embodiments and their salts, solvates and solvates of the salts if the compounds, comprised by formula (I), mentioned below are not already salts, solvates and solvates of the salts.

Depending on their structure, the compounds according to the invention can exist in stereoisomeric forms (enantiomers, diastereomers). Accordingly, the invention comprises the enantiomers or diastereomers and their respective mixtures. From such mixtures of enantiomers and/or diastereomers, it is possible to isolate the stereoisomerically uniform components in a known manner.

If the compounds according to the invention can be present in tautomeric forms, the present invention comprises all tautomeric forms.

In the context of the present invention, preferred salts are physiologically acceptable salts of the compounds according to the invention. The invention also comprises salts which for their part are not suitable for pharmaceutical applications, but which can be used, for example, for isolating or purifying the compounds according to the invention.

Physiologically acceptable salts of the compounds according to the invention include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalene disulphonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of customary bases, such as, by way of example and by way of preference, alkali metal salts (for example sodium salts and potassium salts), alkaline earth metal salts (for example calcium salts and magnesium salts) and ammonium salts, derived from ammonia or organic amines having 1 to 16 carbon atoms, such as, by way of example and by way of preference, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine and N-methylpiperidine.

In the context of the invention, solvates are those forms of the compounds according to the invention which, in solid or liquid state, form a complex by coordination with solvent molecules. Hydrates are a specific form of the solvates where the coordination is with water. In the context of the present invention, preferred solvates are hydrates.

Moreover, the present invention also comprises prodrugs of the compounds according to the invention. The term “prodrugs” includes compounds which for their part may be biologically active or inactive but which, during the time they spend in the body, are converted into compounds according to the invention (for example metabolically or hydrolytically).

In the context of the present invention, unless specified differently, the substituents have the following meanings:

In the context of the invention, (C₁-C₆)-alkyl and (C₁-C₄)-alkyl represent a straight-chain or branched alkyl radical having 1 to 6 and 1 to 4 carbon atoms, respectively. Preference is given to a straight-chain or branched alkyl radical having 1 to 4 carbon atoms. The following radicals may be mentioned by way of example and by way of preference: methyl, ethyl, n-propyl, isopropyl, n-butyl, isobutyl, sec-butyl, tert-butyl, 1-ethylpropyl, n-pentyl and n-hexyl.

In the context of the invention, (C₃-C₇)-cycloalkyl represents a monocyclic cycloalkyl group having 3 to 7 carbon atoms. Preference is given to a cycloalkyl radical having 3 to 6 carbon atoms. The following radicals may be mentioned by way of example and by way of preference: cyclopropyl, cyclobutyl, cyclopentyl, cyclohexyl and cycloheptyl.

In the context of the invention, (C₁-C₆)-alkoxy and (C₁-C₄)-alkoxy represent a straight-chain or branched alkoxy radical having 1 to 6 and 1 to 4 carbon atoms, respectively.

Preference is given to a straight-chain or branched alkoxy radical having 1 to 4 carbon atoms. The following radicals may be mentioned by way of example and by way of preference: methoxy, ethoxy, n-propoxy, isopropoxy, n-butoxy and tert-butoxy.

In the context of the invention, (C₁-C₆)-alkanoyl [(C₁-C₆)-acyl] represents a straight-chain or branched alkyl radical having 1 to 6 carbon atoms which carries a doubly attached oxygen atom in the 1-position and is attached via the 1-position. Preference is given to a straight-chain or branched alkanoyl radical having 1 to 4 carbon atoms. The following radicals may be mentioned by way of example and by way of preference: formyl, acetyl, propionyl, n-butyryl, isobutyryl and pivaloyl.

In the context of the invention, (C₁-C₆)-alkoxycarbonyl represents a straight-chain or branched alkoxy radical having 1 to 6 carbon atoms which is attached via a carbonyl group. Preference is given to a straight-chain or branched alkoxycarbonyl radical having 1 to 4 carbon atoms in the alkoxy group. The following radicals may be mentioned by way of example and by way of preference: methoxycarbonyl, ethoxycarbonyl, n-propoxycarbonyl, isopropoxycarbonyl and tert-butoxycarbonyl.

In the context of the invention, mono-(C₁-C₆)-alkylamino and mono-(C₁-C₄)-alkylamino represent an amino group having a straight-chain or branched alkyl substituent having 1 to 6 and 1 to 4 carbon atoms, respectively. Preference is given to a straight-chain or branched monoalkylamino radical having 1 to 4 carbon atoms. The following radicals may be mentioned by way of example and by way of preference: methylamino, ethylamino, n-propylamino, isopropylamino and tert-butylamino.

In the context of the invention, di-(C₁-C₆)-alkylamino and di-(C₁-C₄)-alkylamino represent an amino group having two identical or different straight-chain or branched alkyl substituents having in each case 1 to 6 and 1 to 4 carbon atoms, respectively. Preference is given to straight-chain or branched dialkylamino radicals having in each case 1 to 4 carbon atoms. The following radicals may be mentioned by way of example and by way of preference: N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

In the context of the invention, (C₃-C₇)-cycloalkylamino represents an amino group having a cycloalkyl substituent which has 3 to 7 carbon atoms. Preference is given to a cycloalkylamino radical having 3 to 6 carbon atoms. The following radicals may be mentioned by way of example and by way of preference: cyclopropylamino, cyclobutylamino, cyclopentylamino, cyclohexylamino and cycloheptylamino.

In the context of the invention, (C₁-C₆)-alkanoylamino represents an amino group having a straight-chain or branched alkanoyl substituent which has 1 to 6 carbon atoms and is attached via the carbonyl group. Preference is given to an alkanoylamino radical having 1 to 4 carbon atoms. The following radicals may be mentioned by way of example and by way of preference: formamido, acetamido, propionamido, n-butyramido and pivaloylamido.

In the context of the invention, (C₁-C₆)-alkoxycarbonylamino represents an amino group having a straight-chain or branched alkoxycarbonyl substituent which has 1 to 6 carbon atoms in the alkoxy radical and is attached via the carbonyl group. Preference is given to an alkoxycarbonylamino radical having 1 to 4 carbon atoms in the alkoxy group. The following radicals may be mentioned by way of example and by way of preference: methoxycarbonylamino, ethoxycarbonylamino, n-propoxycarbonylamino and tert-butoxy-carbonylamino.

In the context of the invention, a 4- to 7-membered heterocycle represents a saturated heterocycle having 4 to 7 ring atoms which contains a ring nitrogen atom and is attached via this ring nitrogen atom and which may contain a further heteroatom from the group consisting of N and O as ring member. Preference is given to a 5- or 6-membered saturated heterocycle which is attached via nitrogen and may contain a further heteroatom from the group consisting of N and O. The following radicals may be mentioned by way of example: azetidinyl, pyrrolidinyl, oxazolidinyl, imidazolidinyl, piperidinyl, piperazinyl, morpholinyl, azepinyl and 1,4-diazepinyl. Particular preference is given to pyrrolidinyl, piperidinyl, piperazinyl and morpholinyl.

In the context of the invention, a 4- to 6-membered saturated or partially unsaturated heterocycle represents a heterocycle having 4 to 6 ring atoms which contains a ring nitrogen atom and is attached via this ring nitrogen atom and which may contain a further heteroatom from the group consisting of N and O and is saturated or contains a double bond. Preference is given to a 5- or 6-membered saturated or partially unsaturated heterocycle which is attached via nitrogen and may contain a further heteroatom from the group consisting of N and O. The following radicals may be mentioned by way of example: azetidinyl, pyrrolidinyl, oxazolidinyl, imidazolidinyl, pyrrolinyl, pyrazolinyl, imidazolinyl, 4-oxazolinyl, isoxazolinyl, piperidinyl, piperazinyl, morpholinyl, tetrahydropyridinyl and tetrahydropyrimidinyl. Particular preference is given to pyrrolidinyl, pyrrolinyl, piperidinyl, piperazinyl and morpholinyl.

In the context of the invention, halogen includes fluorine, chlorine, bromine and iodine. Preference is given to fluorine or chlorine.

If radicals in the compounds according to the invention are substituted, the radicals can, unless specified otherwise, be mono- or polysubstituted. In the context of the present invention, the meanings of radicals which occur more than once are independent of one another. Substitution with one, two or three identical or different substituents is preferred. Very particular preference is given to substitution with one substituent.

Preference is given to compounds of the formula (I) in which

A represents a group of the formula

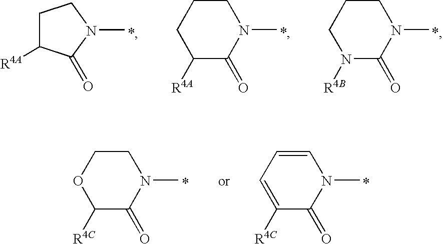

in which
R^{4A} represent hydrogen, hydroxyl, methoxy or amino,
R^{4B} represents methyl or ethyl which may in each case be substituted by hydroxyl, amino, pyrrolidino or cyclopropylamino, or represents amino,
R^{4C} represents hydrogen, methyl or ethyl, where methyl and ethyl may in each case be substituted by hydroxyl, amino, pyrrolidino or cyclopropylamino,
and
* denotes the point of attachment to the phenyl ring,
   Z represents a group of the formula

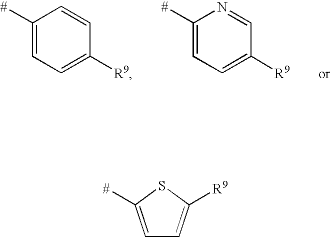

in which
R⁹ represents fluorine, chlorine, methyl or ethynyl
and
# denotes the point of attachment to the carbonyl group,
   R¹ represents hydrogen,
   R² represents hydrogen, fluorine or methyl,
   and
   R³ represents hydrogen or (C₁-C₄)-alkyl,
   and salts, solvates and solvates of the salts thereof.

Particular preference is given to compounds of the formula (I) in which

A represents a group of the formula

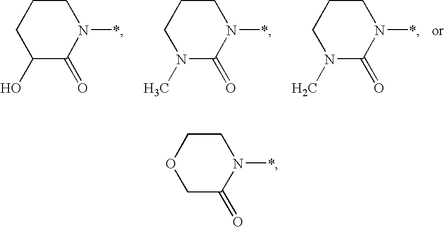

in which * denotes the point of attachment to the phenyl ring,

Z represents a group of the formula

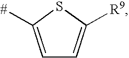

in which

R⁹ represents fluorine, chlorine or methyl

and

# denotes the point of attachment to the carbonyl group,

R¹ represents hydrogen,
R² represents hydrogen, fluorine or methyl,
and
R³ represents hydrogen,
and salts, solvates and solvates of the salts thereof.

The individual radical definitions given in the respective combinations or preferred combinations of radicals may, independently of the particular given combination of radicals, also be replaced by any radical definitions of other combinations.

Very particular preference is given to combinations of two or more of the preferred ranges mentioned above.

The invention furthermore provides a process for preparing the compounds of the formula (I) according to the invention in which R³ represents hydrogen, characterized in that (2S)-3-aminopropane-1,2-diol of the formula (II)

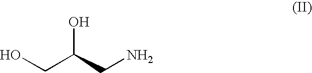

is reacted in an inert solvent in the presence of a base with a compound of the formula (III)

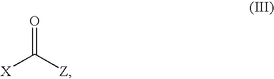

in which Z has the meaning given above and
X represents a suitable leaving group such as, for example, halogen, preferably chlorine,
to give compounds of the formula (IV)

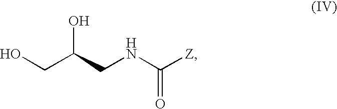

in which Z has the meaning given above,
then converted with the aid of hydrobromic acid in acetic acid, if appropriate with addition of acetic anhydride, into compounds of the formula (V)

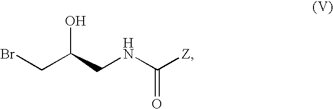

in which Z has the meaning given above,
these are then cyclized in an inert solvent in the presence of a base into compounds of the formula (VI)

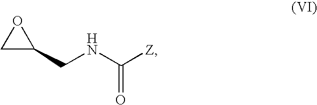

in which Z has the meaning given above,
then reacted in an inert solvent, if appropriate in the presence of a protic acid or Lewis acid, with a compound of the formula (VII)

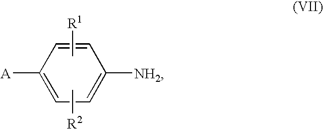

in which A, R¹ and R² have the meanings given above,
to give compounds of the formula (VIII)

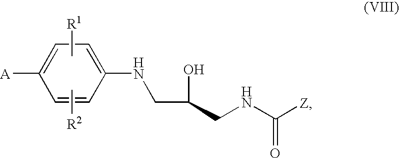

in which A, Z, R¹ and R² have the meanings given above,
and these are then reacted in an inert solvent with cyanogen bromide, if appropriate in the presence of an acid, to give compounds of the formula (I-A)

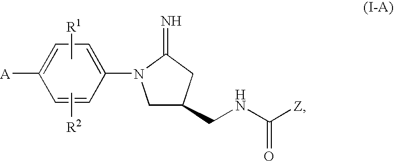

in which A, Z, R¹ and R² have the meanings given above,
and the compounds of the formula (I-A) are, if appropriate, converted with the appropriate (i) solvents and/or (ii) bases or acids into their solvates, salts and/or solvates of the salts.

The compounds of the formula (I) according to the invention in which R³ does not represent hydrogen can be prepared from the compounds of the formula (VIII) analogously to processes known from the literature [cf., for example, for R³=cyano: a) R. Evers, M. Michalik, J. Prakt. Chem. 1991, 333, 699-710; N. Maezaki, A. Furusawa, S. Uchida, T. Tanaka, Tetrahedron 2001, 57, 9309-9316; G. Berecz, J. Reiter, G. Argay, A. Kalman, J. Heterocycl. Chem. 2002, 39, 319-326; b) R. Mohr, A. Buschauer, W. Schunack, Arch. Pharm. (Weinheim Ger.) 1988, 321, 221-227; for R³=alkyl: a) V. A. Vaillancourt et al., J. Med. Chem. 2001, 44, 1231-1248; b) F. B. Dains et al., J. Amer. Chem. Soc. 1925, 47, 1981-1989; J. Amer. Chem. Soc. 1922, 44, 2637-2643 and T. Shibanuma, M. Shiono, T. Mukaiyama, Chem. Lett. 1977, 575-576; see also Synthesis schemes 2 and 3].

If appropriate, the compounds according to the invention can also be prepared by further conversions of functional groups of individual substituents, in particular the substituents listed under R¹, R², R⁴, R⁶ and R⁷, starting with the compounds of the formula (I) obtained by the above process. These conversions are carried out by customary methods and include, for example, reactions such as alkylation, amination, acylation, esterification, ester cleavage, amide formation, oxidation or reduction and also the introduction and removal of temporary protective groups.

Inert solvents for process step (II)+(III)→(IV) are, for example, halogenated hydrocarbons, such as dichloromethane, trichloromethane, carbon tetrachloride, tri-chloroethane, tetrachloroethane, 1,2-dichloroethane or trichloroethylene, ethers, such as diethyl ether, dioxane, tetrahydrofuran, 2-methyltetrahydrofuran, glycol dimethyl ether or diethylene glycol dimethyl ether, hydrocarbons, such as benzene, xylene, toluene, hexane, cyclohexane or mineral oil fractions, or other solvents, such as acetone, dimethylformamide, dimethyl sulphoxide, N,N′-dimethylpropyleneurea (DMPU), N-methylpyrrolidone (NMP), acetonitrile, pyridine or else water. It is also possible to use mixtures of the solvents mentioned. Preference is given to using a mixture of water, 2-methyltetrahydrofuran and toluene.

In this process step, (2S)-3-aminopropane-1,2-diol (II) can be employed as free base or as acid addition salt; preference is given to using the hydrochloride.

Suitable bases for the process step (II)+(III)→(IV) are customary inorganic or organic bases. These include in particular alkali metal bicarbonates, such as sodium or potassium bicarbonate, alkali metal or alkaline earth metal carbonates, such as lithium carbonate, sodium carbonate, potassium carbonate, calcium carbonate or caesium carbonate, or organic amines, such as triethylamine, N-methylmorpholine, N-methylpiperidine, N,N-diisopropylethylamine or pyridine. Preference is given to using sodium bicarbonate.

Here, the base is employed in an amount of from 1 to 3 mol, preferably in an amount of from 1 to 2 mol, per mole of the compound of the formula (II) or its hydrochloride. The reaction is generally carried out in a temperature range of from 0° C. to +50° C., preferably from +5° C. to +30° C.

The reaction (IV)→(V) is carried out using 1 to 5, preferably 3 to 5, equivalents of aqueous hydrobromic acid in acetic acid, if appropriate with addition of acetic anhydride. The reaction is generally carried out in a temperature range of from +20° C. to +100° C., preferably from +50° C. to +80° C.

Inert solvents for the process step (V)→(VI) are, for example, halogenated hydrocarbons, such as dichloromethane, trichloromethane, carbon tetrachloride, trichloroethane, tetra-chloroethane, 1,2-dichloroethane or trichloroethylene, ethers, such as diethyl ether, dioxane, tetrahydrofuran, 2-methyltetrahydrofuran, glycol dimethyl ether or diethylene-glycol dimethyl ether, hydrocarbons, such as benzene, xylene, toluene, hexane, cyclohexane or mineral oil fractions, or other solvents, such as dimethylformamide, dimethyl sulphoxide, N,N′-dimethylpropyleneurea (DMPU), N-methylpyrrolidone (NMP) or acetonitrile. It is also possible to use mixtures of the solvents mentioned. Preference is given to using dichloromethane or tetrahydrofuran.

Suitable bases for the cyclization (V)→(VI) are customary inorganic bases. These include in particular alkali metal bicarbonates, such as sodium bicarbonate or potassium bicarbonate, or alkali metal or alkaline earth metal carbonates, such as lithium carbonate, sodium carbonate, potassium carbonate, calcium carbonate or caesium carbonate. Preference is given to using potassium carbonate.

Here, the base is employed in an amount of from 1 to 5 mol, preferably in an amount of from 3 to 5 mol, per mole of the compound of the formula (V). The reaction is generally carried out in a temperature range of from 0° C. to +50° C., preferably from +10° C. to +30° C.

Inert solvents for the process step (VI)+(VII)→(VIII) are, for example, alcohols, such as methanol, ethanol, n-propanol, isopropanol, n-butanol or tert-butanol, ethers, such as diethyl ether, dioxane, tetrahydrofuran, 2-methyltetrahydrofuran, glycol dimethyl ether or diethylene glycol dimethyl ether, or other solvents, such as acetone, dimethylformamide, dimethyl sulphoxide, N,N′-dimethylpropyleneurea (DMPU), N-methylpyrrolidone (NMP), acetonitrile or else water. It is also possible to use mixtures of the solvents mentioned. Preference is given to using dioxane, tetrahydrofuran, ethanol or mixtures thereof with water.

The process step (VI)+(VII)→(VIII) can optionally also be carried out with addition of a catalytic amount of a protic acid, such as, for example, p-toluenesulphonic acid, or a Lewis acid, such as, for example, ytterbium(III) trifluoromethanesulphonate.

The reaction (VI)+(VII)→(VIII) is generally carried out in a temperature range of from 0° C. to +100° C., preferably from +20° C. to +80° C.

Inert solvents for the process step (VIII)→(I-A) are, for example, halogenated hydrocarbons, such as dichloromethane, trichloromethane, carbon tetrachloride, tri-chloroethane, tetrachloroethane or 1,2-dichloroethane, ethers, such as diethyl ether, dioxane or tetrahydrofuran, hydrocarbons, such as benzene, xylene, toluene, hexane, cyclohexane or mineral oil fractions, or other solvents, such as acetonitrile. It is also possible to use mixtures of the solvents mentioned. Preference is given to using tetrahydrofuran or acetonitrile.

Advantageously, the process step (VIII)→(I-A) can be carried out with addition of a strong inorganic or organic acid. These include in particular acids such as hydrogen fluoride, hydrogen chloride, hydrogen bromide, methanesulphonic acid, trifluoromethanesulphonic acid or trifluoroacetic acid.

The reaction (VIII)→(I-A) is generally carried out in a temperature range of from −20° C. to +50° C., preferably from 0° C. to +40° C.

All process steps can be carried out at atmospheric pressure, elevated pressure or reduced pressure (for example from 0.5 to 5 bar). In general, the reactions are carried out at atmospheric pressure.

The compounds of the formulae (II), (III) and (VII) are commercially available, known from the literature, or they can be prepared analogously to processes known from the literature.

The preparation of the compounds according to the invention can be illustrated by the synthesis schemes below:

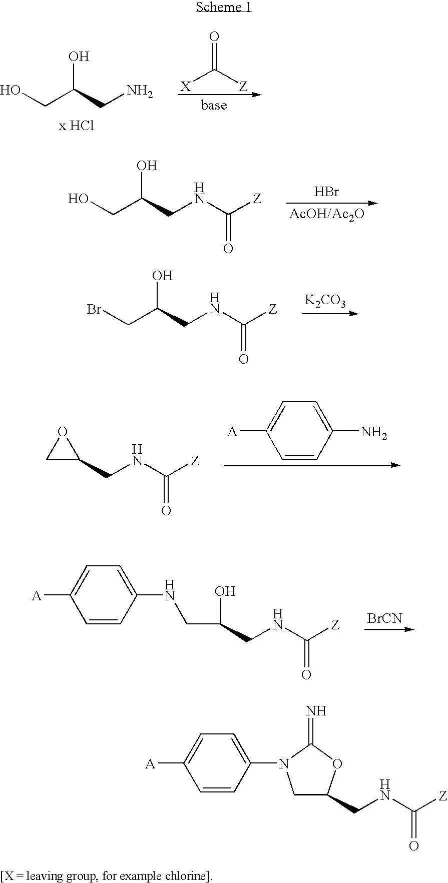

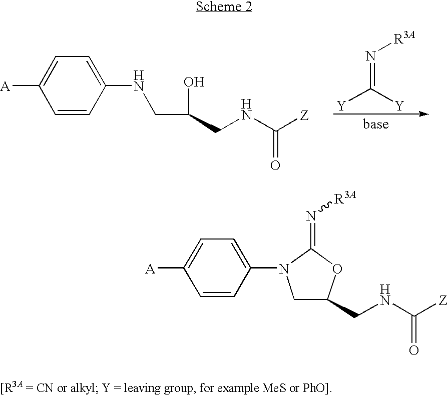

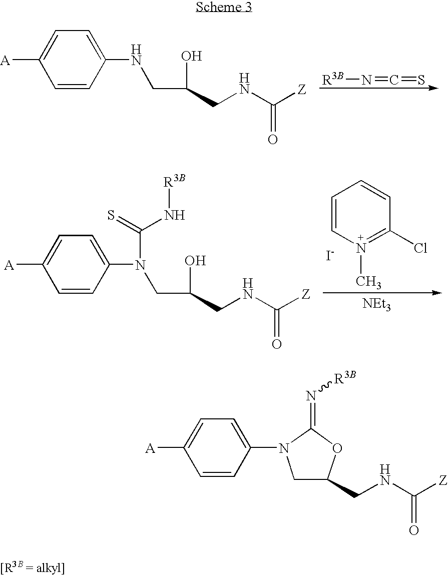

The compounds according to the invention have an unforeseeable useful pharmacological activity spectrum, in particular high efficacy.

Accordingly, they are suitable for use as medicaments for the treatment and/or prophylaxis of diseases in humans and animals.

The compounds according to the invention are selective inhibitors of blood coagulation factor Xa which act in particular as anticoagulants.

In addition, the compounds according to the invention have favourable physicochemical properties, such as, for example, good solubility in water and physiological media, which is advantageous for their therapeutic application.

The present invention furthermore provides the use of the compounds according to the invention for the treatment and/or prophylaxis of disorders, preferably thromboembolic disorders and/or thromboembolic complications.

For the purposes of the present invention, “thromboembolic disorders” include in particular disorders such as ST-elevation mycardial infarction (STEMI) or non-ST-elevation mycardial infarction (non-STEMI), stable angina pectoris, unstable angina pectoris, reocclusions and restenoses after coronary interventions such as angioplasty or aortocoronary bypass, peripheral areterial occlusive diseases, pulmonary embolisms, deep vein thromboses and kidney vein thromboses, transitory ischaemic attacks and also thrombotic and thromboembolic stroke.

Accordingly, the substances are also suitable for preventing and treating cardiogenic thromboembolisms, such as, for example, brain ischaemias, stroke and systemic thromboembolisms and ischaemias, in patients having acute, intermittent or persistent cardioarrhythmias, such as, for example, atrial fibrillation, and those undergoing cardioversion, furthermore patients having heart valve disorders or having artificial heart valves. In addition, the compounds according to the invention are suitable for treating disseminated intravascular coagulation (DIC).

Thromboembolic complications furthermore occur during microangiopathic haemolytic anaemias, extracorporeal blood circulation, such as haemodialysis, and in connection with heart valve prostheses.

Moreover, the compounds according to the invention are also suitable for the prophylaxis and/or treatment of atherosclerotic vascular disorders and inflammatory disorders, such as rheumatic disorders of the locomotor apparatus, and in addition also for the prophylaxis and/or treatment of Alzheimer's disease. Moreover, the compounds according to the invention can be used for inhibiting tumour growth and formation of metastases, for microangiopathies, age-related macular degeneration, diabetic retinopathy, diabetic nephropathy and other microvascular disorders, and also for the prevention and treatment of thromboembolic complications, such as, for example, venous thromboembolisms, in tumour patients, in particular patients undergoing major surgical interventions or chemo- or radiotherapy.

The compounds according to the invention can additionally also be used for preventing coagulation ex vivo, for example for preserving blood and plasma products, for cleaning/pretreating catheters and other medical tools and instruments, for coating synthetic surfaces of medical tools and instruments used in vivo or ex vivo or for biological samples comprising factor Xa.

The present invention furthermore provides the use of the compounds according to the invention for the treatment and/or prophylaxis of disorders, in particular the disorders mentioned above.

The present invention furthermore provides the use of the compounds according to the invention for preparing a medicament for the treatment and/or prophylaxis of disorders, in particular the disorders mentioned above.

The present invention furthermore provides a method for the treatment and/or prophylaxis of disorders, in particular the disorders mentioned above, using an anticoagulatory effective amount of the compound according to the invention.

The present invention furthermore provides a method for preventing blood coagulation in vitro, in particular in banked blood or biological samples comprising factor Xa, which method is characterized in that an anticoagulatory effective amount of the compound according to the invention is added.

The present invention furthermore provides medicaments comprising a compound according to the invention and one or more further active compounds, in particular for the treatment and/or prophylaxis of the disorders mentioned above. The following compounds may be mentioned by way of example and by way of preference as active compounds suitable for combinations:
lipid-lowering agents, in particular HMG-CoA (3-hydroxy-3-methylglutaryl-coenzyme A) reductase inhibitors;
coronary therapeutics/vasodilators, in particular ACE (angiotensin converting enzyme) inhibitors; AII (angiotensin II) receptor antagonists; β-adrenoceptor antagonists; alpha-1-adrenoceptor antagonists; diuretics; calcium channel blockers; substances which cause an increase in the cyclic guanosine monophosphate (cGMP) concentration such as, for example, stimulators of soluble guanylate cyclase;
plasminogen activators (thrombolytics/fibrinolytics) and compounds enhancing thrombolysis/fibrinolysis, such as inhibitors of the plasminogen activator inhibitor (PAI inhibitors) or inhibitors of the thrombin-activated fibrinolysis inhibitor (TAFI inhibitors);
anticoagulants;
platelet aggregation inhibiting substances (platelet aggregation inhibitors, thrombocyte aggregation inhibitors);
fibrinogen receptor antagonists (glycoprotein-IIb/IIIa antagonists).

The present invention furthermore provides medicaments comprising at least one compound according to the invention, usually together with one or more inert nontoxic pharmaceutically acceptable auxiliaries, and their use for the purposes mentioned above.

The compounds according to the invention can act systemically and/or locally. For this purpose, they can be administered in a suitable way, such as, for example, by the oral, parenteral, pulmonary, nasal, sublingual, lingual, buccal, rectal, dermal, transdermal, conjunctival or otic route, or as implant or stent.

For these administration routes, it is possible to administer the compounds according to the invention in suitable administration forms.

Suitable for oral administration are administration forms which work as described in the prior art and deliver the compounds according to the invention rapidly and/or in modified form, which comprise the compounds according to the invention in crystalline and/or amorphous and/or dissolved form, such as, for example, tablets (uncoated and coated tablets, for example tablets provided with enteric coatings or coatings whose dissolution is delayed or which are insoluble and which control the release of the compound according to the invention), tablets which rapidly decompose in the oral cavity, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (for example intravenously, intraarterially, intracardially, intraspinally or intralumbarly) or with inclusion of absorption (for example intramuscularly, subcutaneously, intracutaneously, percutaneously or intraperitoneally). Administration forms suitable for parenteral administration are, inter alia, preparations for injection and infusion in the form of solutions, suspensions, emulsions, lyophilizates or sterile powders.

Examples suitable for other administration routes are pharmaceutical forms for inhalation (inter alia powder inhalers, nebulizers), nasal drops/solutions/sprays; tablets to be administered lingually, sublingually or buccally, films/wafers or capsules, suppositories, preparations for the eyes or ears, vaginal capsules, aqueous suspensions (lotions, shaking mixtures), lipophilic suspensions, ointments, creams, transdermal therapeutic systems, (e.g. patches), milk, pastes, foams, dusting powders, implants or stents.

Preference is given to oral or parenteral administration, in particular oral administration.

The compounds according to the invention can be converted into the stated administration forms. This can take place in a manner known per se by mixing with inert, nontoxic, pharmaceutically suitable auxiliaries. These auxiliaries include, inter alia, carriers (for example microcrystalline cellulose, lactose, mannitol), solvents (for example liquid polyethylene glycols), emulsifiers and dispersants or wetting agents (for example sodium dodecyl sulphate, polyoxysorbitan oleate), binders (for example polyvinylpyrrolidone), synthetic and natural polymers (for example albumin), stabilizers (for example antioxidants, such as, for example, ascorbic acid), colorants (for example inorganic pigments, such as, for example, iron oxides) and flavour- and/or odour-masking agents.

In general, it has proved advantageous to administer on parenteral administration amounts of from about 0.001 to 1 mg/kg, preferably from about 0.01 to 0.5 mg/kg, of body weight to achieve effective results. The dosage on oral administration is from about 0.01 to 100 mg/kg, preferably about 0.01 to 20 mg/kg, and very particularly preferably 0.1 to 10 mg/kg, of body weight.

It may nevertheless be necessary, where appropriate, to deviate from the amounts mentioned, depending on the body weight, the administration route, the individual response to the active compound, the mode of preparation and the time or interval over which administration takes place. Thus, in some cases it may be sufficient to make do with less than the aforementioned minimal amount, whereas in other cases the upper limit mentioned must be exceeded. In the event of administration of larger amounts, it may be advisable to divide these into a plurality of individual doses over the day.

The invention is illustrated by the working examples below. The invention is not limited to the examples.

The percentage data in the following tests and examples are percentages by weight unless otherwise indicated; parts are parts by weight. Solvent ratios, dilution ratios and concentration data of liquid/liquid solutions are in each case based on volume.

### A. EXAMPLES

### Abbreviations and Acronyms

Ac acetyl
DMSO dimethyl sulphoxide
ESI electrospray ionization (in MS)
Et ethyl
h hour(s)
HPLC high pressure, high performance liquid chromatography
LC-MS liquid chromatography-coupled mass spectroscopy
Me methyl
min minute(s)
MS mass spectroscopy
NMR nuclear magnetic resonance spectroscopy
Ph phenyl
RP reverse phase (in HPLC)
Rₜ retention time (in HPLC)
THF tetrahydrofuran

### LC-MS and HPLC Methods:

### Method 1:

MS instrument: Micromass ZQ; HPLC instrument; Waters Alliance 2795; column: Phenomenex Synergi 2μ Hydro-RP Mercury 20 mm×4 mm; mobile phase A: 1 l of water+0.5 ml of 50% strength formic acid, mobile phase B: 1 l of acetonitrile+0.5 ml of 50% strength formic acid; gradient: 0.0 min 90% A→2.5 min 30% A→3.0 min 5% A→4.5 min 5% A; flow rate: 0.0 min 1 ml/min, 2.5 min/3.0 min/4.5 min 2 ml/min; oven: 50° C.; UV detection: 210 nm.

### Method 2:

Instrument: HP 1100 with DAD detection; column: Kromasil 100 RP-18, 60 mm×2.1 mm, 3.5 μm; mobile phase A: 5 ml of HClO₄ (70%)/l of water, mobile phase B: acetonitrile; gradient: 0 min 2% B→0.5 min 2% B→4.5 min 90% B→9 min 0% B→9.2 min 2% B→10 min 2% B; flow rate: 0.75 ml/min; column temperature: 30° C.; UV detection: 210 nm.

### Starting Materials:

### Example 1A

### 5-Chlorothiophene-2-carbonyl chloride

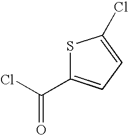

The title compound is prepared by reacting 5-chlorothiophene-2-carboxylic acid with thionyl chloride, see R. Aitken et al., Arch. Pharm. (Weinheim Ger.) 1998, 331, 405-411.

### Example 2A

### 4-(4-Aminophenyl)morpholin-3-one

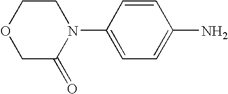

The title compound is prepared by reacting 4-fluoronitrobenzene with morpholin-3-one [J.-M. Lehn, F. Montavon, Helv. Chim. Acta 1976, 59, 1566-1583] and subsequently reducing the resulting 4-(4-nitrophenyl)morpholin-3-one (see WO 01/47919, starting materials I and II, pp. 55-57).

### Example 3A

### 5-Chloro-N-[(2S)-2-oxiranylmethyl]-2-thiophenecarboxamide

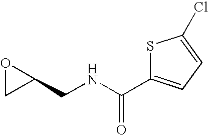

The title compound is prepared as described in WO 2004/101557 (Example 6A) by (i) acylation of (2S)-3-aminopropane-1,2-diol hydrochloride with 5-chlorothiophene-2-carbonyl chloride in the presence of sodium bicarbonate as base, (ii) hydroxy-bromine exchange with the aid of hydrobromic acid in acetic acid/acetic anhydride and (iii) epoxide formation in the presence of potassium carbonate as base.

### WORKING EXAMPLES

### Example 1

### 5-Chloro-N-({(5S)-2-imino-3-[4-(3-oxomorpholin-4-yl)phenyl]-1,3-oxazolidin-5-yl}methyl)-thiophene-2-carboxamide

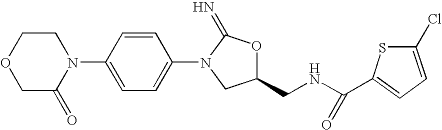

### Step a): 5-Chloro-N-((2R)-2-hydroxy-3-{[4-(3-oxo-4-morpholinyl)phenyl]amino}-propyl)-2-thiophenecarboxamide

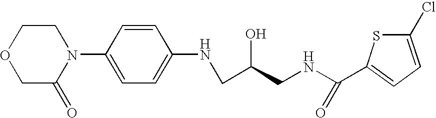

6.18 g (32 mmol) of 4-(4-aminophenyl)morpholin-3-one (Example 2A) and 7.00 g (32 mmol) of 5-chloro-N-[(2S)-2-oxiranylmethyl]-2-thiophenecarboxamide (Example 3A) are suspended in 130 ml of ethanol/water (9:1) and stirred at 75° C. overnight (formation of a solution). The solution is cooled in an ice-bath, and the resulting white precipitate is filtered off, washed with diethyl ether and dried under high vacuum. This gives 4.98 g of the title compound. Concentration of the mother liquor, another addition of 3.5 g (16 mmol) of 5-chloro-N-[(2S)-2-oxiranylmethyl]-2-thiophenecarboxamide in 50 ml of ethanol/water (9:1), more stirring at 75° C. overnight and filtration of the precipitate obtained after cooling in an ice-bath gives another 3.44 g of the title compound.

Yield: 8.42 g in total (62% of theory)

LC-MS (method 1): Rₜ=1.46 min;

MS (ESIpos): m/z=410 [M+H]⁺;

¹H-NMR (300 MHz, DMSO-d₆): δ=8.60 (t, 1H), 7.69 (d, 1H), 7.18 (d, 1H), 7.02 (d, 2H), 6.59 (d, 2H), 5.65 (t, 1H), 5.08 (d, 1H), 4.13 (s, 2H), 3.91 (dd, 2H), 3.87-3.74 (m, 1H), 3.59 (m, 2H), 3.30-2.90 (m, 4H).

Step b): 5-Chloro-N-({(5S)-2-imino-3-[4-(3-oxomorpholin-4-yl)phenyl]-1,3-oxazolidin-5-yl}methyl)thiophene-2-carboxamide

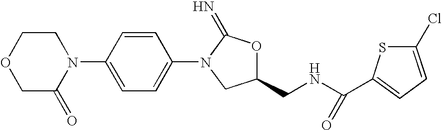

Under argon and at room temperature, 700 mg (1.71 mmol) of 5-chloro-N-((2R)-2-hydroxy-3-{[4-(3-oxo-4-morpholinyl)phenyl]amino}propyl)-2-thiophenecarboxamide from step a) are stirred in 13.7 ml of a 5-molar solution of cyanogen bromide (68.3 mmol) in THF for 6 h. The reaction mixture is concentrated and the residue (690 mg) is purified by preparative HPLC [column: YMC Gel ODS-AQ S-11 μm; mobile phase: acetonitrile/0.2% strength trifluoroacetic acid 35:65]. This gives 491 mg (89% of theory) of the title compound as trifluoroacetate salt. The free base is released by stirring with saturated aqueous sodium bicarbonate solution in THF, and the solution is then extracted with dichloromethane. The combined organic phases are dried over sodium sulphate, filtered and concentrated under reduced pressure. This gives 282 mg (38% of theory) of the title compound as free base.

HPLC (method 2): Rₜ=3.58 min;

MS (ESIpos): m/z=435 [M+H]⁺;

¹H-NMR (400 MHz, DMSO-d₆): δ=8.91 (t, 1H), 7.79 (d, 2H), 7.67 (d, 1H), 7.33 (d, 2H), 7.19 (d, 1H), 6.15 (s, 1H), 4.78-4.69 (m, 1H), 4.19 (s, 2H), 4.16-4.08 (m, 1H), 3.97 (dd, 2H), 3.79 (m, 1H), 3.69 (dd, 2H), 3.58-3.50 (m, 2H).

### B. EVALUATION OF THE PHARMACOLOGICAL ACTIVITY

The compounds according to the invention act in particular as selective inhibitors of blood coagulation factor Xa and do not, or only at significantly higher concentrations, inhibit other serine proteases, such as plasmin or trypsin.

Inhibitors of blood coagulation factor Xa are referred to as being “selective” if the IC₅₀ values for factor Xa inhibition are smaller by a factor of at least 100 compared with the IC₅₀ values for the inhibition of other serine proteases, in particular plasmin and trypsin, where, with a view to the test methods for selectivity, reference is made to the test methods described below of Examples B.a.1) and B.a.2).

The advantageous pharmacological properties of the compounds according to the invention can be determined by the following methods:

### a) Test Description (In Vitro)

### a.1) Determination of the Factor Xa Inhibition:

The enzymatic inhibition of human factor Xa (FXa) is measured using the conversion of a chromogenic substrate specific for FXa. Factor Xa cleaves p-nitroaniline from the chromogenic substrate. The determinations are carried out in microtitre plates as follows:

The test substances, in various concentrations, are dissolved in DMSO and incubated for 10 minutes at 25° C. with human FXa (0.5 nmol/l dissolved in 50 mmol/l of Tris buffer [C,C,C-tris(hydroxymethyl)aminomethane], 150 mmol/l of NaCl, 0.1% BSA [bovine serum albumin], pH=8.3). Pure DMSO is used as control. The chromogenic substrate (150 μmol/l Pefachrome® FXa from Pentapharm) is then added. After an incubation time of 20 minutes at 25° C., the extinction at 405 nm is determined. The extinctions of the test mixtures containing the test substance are compared with the control mixtures without test substance, and the IC₅₀ values are calculated from these data.

In this test, working example 1 shows an IC₅₀ of 5.4 nM.

### a.2) Determination of the Selectivity:

To assess selective FXa inhibition, the test substances are examined for their inhibition of other human serine proteases such as trypsin and plasmin. To determine the enzymatic activity of trypsin (500 mU/ml) and plasmin (3.2 nmol/l), these enzymes are dissolved in Tris buffer (100 mmol/l, 20 mmol/l CaCl₂, pH=8.0) and incubated with test substance or solvent for 10 minutes. The enzymatic reaction is then started by adding the corresponding specific chromogenic substrates (Chromozym Trypsin® and Chromozym Plasmin®; from Roche Diagnostics) and the extinction at 405 nm is determined after 20 minutes. All determinations are carried out at 37° C. The extinctions of the test mixtures containing test substance are compared with the control samples without test substance, and the IC₅₀ values are calculated from these data.

In this test, working example 1 shows in each case an IC₅₀ of >10 μM.

### a.3) Determination of the Anticoagulant Action:

The anticoagulant action of the test substances is determined in vitro in human and rabbit plasma. To this end, blood is drawn off in a mixing ratio of sodium citrate/blood of 1:9 using a 0.11 molar sodium citrate solution as receiver. Immediately after the blood has been drawn off, it is mixed thoroughly and centrifuged at about 2500 g for 10 minutes. The supernatant is pipetted off. The prothrombin time (PT, synonyms: thromboplastin time, quick test) is determined in the presence of varying concentrations of test substance or the corresponding solvent using a commercial test kit (Hemoliance® RecombiPlastin, from Instrumentation Laboratory). The test compounds are incubated with the plasma at 37° C. for 3 minutes. Coagulation is then started by addition of thromboplastin, and the time when coagulation occurs is determined. Concentration of test substance which effects a doubling of the prothrombin time is determined.

### b) Determination of the Antithrombotic Activity (In Vivo)

### b.1) Arteriovenous Shunt Model (Rabbit):

Fasting rabbits (strain: Esd: NZW) are anaesthetized by intramuscular administration of Rompun/Ketavet

solution (5 mg/kg and 40 mg/kg, respectively). Thrombus formation is initiated in an arteriovenous shunt in accordance with the method described by C. N. Berry et al. [Semin. Thromb. Hemost. 1996, 22, 233-241]. To this end, the left jugular vein and the right carotid artery are exposed. The two vessels are connected by an extracorporeal shunt using a vein catheter of a length of 10 cm. In the middle, this catheter is attached to a further polyethylene tube (PE 160, Becton Dickenson) of a length of 4 cm which contains a roughened nylon thread which has been arranged to form a loop, to form a thrombogenic surface. The extracorporeal circulation is maintained for 15 minutes. The shunt is then removed and the nylon thread with the thrombus is weighed immediately. The weight of the nylon thread on its own was determined before the experiment was started. Before extracorporeal circulation is set up, the test substances are administered either intravenously via an ear vein or orally using a pharyngeal tube.

### C. WORKING EXAMPLES OF PHARMACEUTICAL COMPOSITIONS

The compounds according to the invention can be converted into pharmaceutical preparations in the following ways:

### Tablet:

### Composition:

100 mg of the compound according to the invention, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg. Diameter 8 mm, radius of curvature 12 mm.

### Production:

The mixture of the compound according to the invention, lactose and starch is granulated with a 5% strength solution (m/m) of the PVP in water. The granules are dried and then mixed with the magnesium stearate for 5 minutes. This mixture is compressed using a conventional tablet press (see above for the dimensions of the tablet). A compressive force of 15 kN is used as a guideline for the compression.

Suspension which can be Administered Orally:

### Composition:

1000 mg of the compound according to the invention, 1000 mg of ethanol (96%), 400 mg of Rhodigel® (xanthan gum from FMC, Pennsylvania, USA) and 99 g of water.

10 ml of oral suspension correspond to a single dose of 100 mg of the compound according to the invention.

### Production:

The Rhodigel is suspended in ethanol, and the compound according to the invention is added to the suspension. The water is added while stirring. The mixture is stirred for about 6 h until the swelling of the Rhodigel is complete.

Solution which can be Administered Orally:

### Composition:

500 mg of the compound according to the invention, 2.5 g of polysorbate and 97 g of polyethylene glycol 400.20 g of oral solution correspond to a single dose of 100 mg of the compound according to the invention.

### Production:

The compound according to the invention is suspended in the mixture of polyethylene glycol and polysorbate with stirring. Stirring is continued until the compound according to the invention has dissolved completely.

i.v. Solution:

The compound according to the invention is, at a concentration below saturation solubility, dissolved in a physiologically acceptable solvent (for example isotonic saline, glucose solution 5% and/or PEG 400 solution 30%). The solution is subjected to sterile filtration and filled into sterile and pyrogen-free injection containers.

1. Compound of the formula (I) 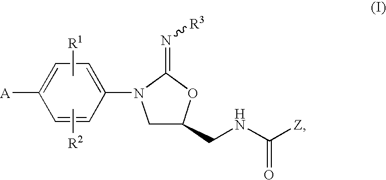
in which
A represents a group of the formula 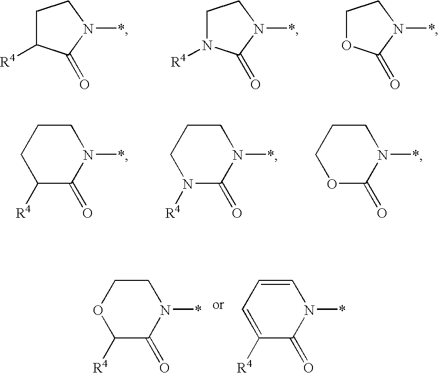
in which
R⁴ represents hydrogen, (C₁-C₆)-alkyl, (C₃-C₇)-cycloalkyl, hydroxy, (C₁-C₆)-alkoxy, amino, mono- or di-(C₁-C₆)-alkylamino, (C₃-C₇)-cycloalkylamino, (C₁-C₆)-alkanoylamino or (C₁-C₆)-alkoxy-carbonylamino, where
(C₁-C₆)-alkyl, (C₁-C₆)-alkoxy, mono- and di-(C₁-C₆)-alkylamino for their part may in each case be substituted by hydroxyl, (C₁-C₄)-alkoxy, amino, mono- or di-(C₁-C₄)-alkylamino, (C₃-C₇)-cycloalkylamino or a 4- to 7-membered saturated heterocycle which is attached via a nitrogen atom and which may contain a ring member from the group consisting of N—R⁵ and O, where
R⁵ represents hydrogen or (C₁-C₄)-alkyl,
and * denotes the point of attachment to the phenyl ring,
or
A represents a group of the formula —C(═O)—NR⁶R⁷, where
R⁶ and R⁷ are identical or different and independently of one another are (C₁-C₆)-alkyl or (C₃-C₇)-cycloalkyl
or
R⁶ and R⁷ together with the nitrogen atom to which they are attached form a 4- to 6-membered saturated or partially unsaturated heterocycle which may contain one ring member from the group consisting of N—R⁸ and O and which may be substituted by (C₁-C₆)-alkyl, hydroxy, (C₁-C₆)-alkoxy, oxo, amino, mono- or di-(C₁-C₆)-alkylamino, where
R⁸ represents hydrogen or (C₁-C₆)-alkyl,
where for their part all (C₁-C₆)-alkyl groups mentioned may be substituted by hydroxyl, (C₁-C₄)-alkoxy, amino, mono- or di-(C₁-C₄)-alkylamino or (C₃-C₇)-cycloalkylamino,
Z represents phenyl, pyridyl, pyrimidinyl, pyrazinyl, thienyl, furyl or pyrrolyl which may in each case be mono- or disubstituted by identical or different substituents selected from the group consisting of halogen, cyano, (C₁-C₄)-alkyl, which for its part may be substituted by hydroxyl or amino, ethynyl, cyclopropyl and amino,
R¹ and R² are identical or different and independently of one another represent hydrogen, halogen, cyano, (C₁-C₄)-alkyl, cyclopropyl, trifluoromethyl, hydroxyl, (C₁-C₄)-alkoxy, trifluoromethoxy, amino, mono- or di-(C₁-C₄)-alkyl-amino, where
(C₁-C₄)-alkyl and (C₁-C₄)-alkoxy for their part may in each case be substituted by hydroxyl, (C₁-C₄)-alkoxy, amino, mono- or di-(C₁-C₄)-alkylamino or (C₃-C₇)-cycloalkylamino,
and
R³ represents hydrogen, (C₁-C₆)-alkyl or cyano,
and salts, solvates and solvates of the salts thereof.

2. A compound of the formula (I) according to claim 1 in which
A represents a group of the formula 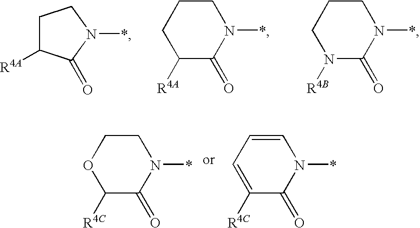
in which
R^{4A} represent hydrogen, hydroxyl, methoxy or amino,
R^{4B} represents methyl or ethyl which may in each case be substituted by hydroxyl, amino, pyrrolidino or cyclopropylamino, or represents amino,
R^{4C} represents hydrogen, methyl or ethyl, where methyl and ethyl may in each case be substituted by hydroxyl, amino, pyrrolidino or cyclopropylamino,
and
* denotes the point of attachment to the phenyl ring,
Z represents a group of the formula 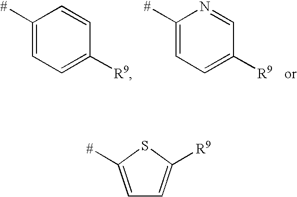
in which
R⁹ represents fluorine, chlorine, methyl or ethynyl
and
# denotes the point of attachment to the carbonyl group,
R¹ represents hydrogen,
R² represents hydrogen, fluorine or methyl,
and
R³ represents hydrogen or (C₁-C₄)-alkyl,
and salts, solvates and solvates of the salts thereof.

3. A compound of the formula (I) according to claim 1 in which
A represents a group of the formula 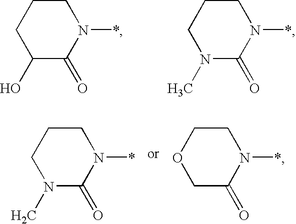
in which * denotes the point of attachment to the phenyl ring,
Z represents a group of the formula 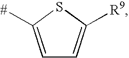
in which
R⁹ represents fluorine, chlorine or methyl
and
# denotes the point of attachment to the carbonyl group,
R¹ represents hydrogen,
R² represents hydrogen, fluorine or methyl,
and
R³ represents hydrogen,
and salts, solvates and solvates of the salts thereof.

4. A method for preparing compounds of the formula (I) as defined in claim 1 in which R³ represents hydrogen, characterized in that (2S)-3-aminopropane-1,2-diol of the formula (II) 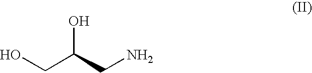
is reacted in an inert solvent in the presence of a base with a compound of the formula (III) 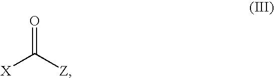
in which Z has the meaning given in claim 1 and
X represents a suitable leaving group such as, for example, halogen, preferably chlorine,
to give compounds of the formula (IV) 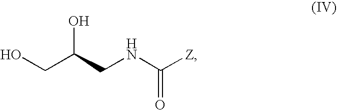
in which Z has the meaning given above,
then converted with the aid of hydrobromic acid in acetic acid, if appropriate with addition of acetic anhydride, into compounds of the formula (V) 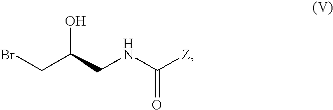
in which Z has the meaning given above,
these are then cyclized in an inert solvent in the presence of a base into compounds of the formula (VI) 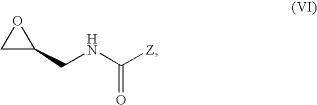
in which Z has the meaning given above,
then reacted in an inert solvent, if appropriate in the presence of a protic acid or Lewis acid, with a compound of the formula (VII) 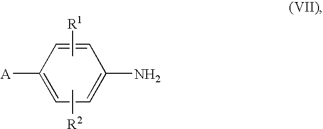
in which A, R¹ and R² have the meanings given in claim 1, to give compounds of the formula (VIII) 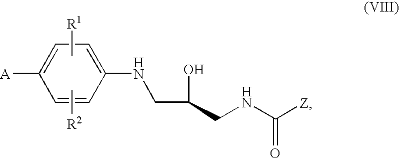
in which A, Z, R¹ and R² have the meanings given above,
and these are then reacted in an inert solvent with cyanogen bromide, if appropriate in the presence of an acid, to give compounds of the formula (I-A) 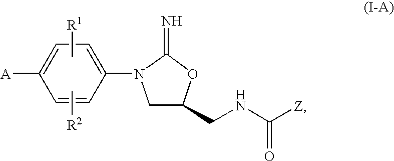
in which A, Z, R¹ and R² have the meanings given above,
and the compounds of the formula (I-A) are, if appropriate, converted with the appropriate (i) solvents and/or (ii) bases or acids into their solvates, salts and/or solvates of the salts.

5. A compound of the formula (I) as defined in claim 1 for the treatment and/or prophylaxis of diseases.

6. (canceled)

7. (canceled)

8. A pharmaceutical composition comprising a compound of the formula (I) as defined in claim 1 in combination with an inert non-toxic, pharmaceutically suitable auxiliary.

9. The pharmaceutical composition of claim 8 further comprising a second active compound.

10. The pharmaceutical composition of claim 8 for the treatment and/or prophylaxis of thromboembolic disorders.

11. A method for the treatment and/or prophylaxis of thromboembolic disorders in humans and animals, comprising administering an anticoagulatory effective amount of at least one compound of the formula (I) as defined in claim 1.

12. A method for preventing blood coagulation in vitro, comprising administering an anticoagulatory effective amount of a compound of the formula (I) as defined in claim 1 is added.

13. A method for the treatment and/or prophylaxis of thromboembolic disorders in humans and animals, comprising administering an anticoagulatory effective amount of a pharmaceutical composition of claim 8.
